(19) United States
(12) Patent Application Publication
(10) Pub. No.: US 2010/0323039 A1
(43) Pub. Date: Dec. 23, 2010

(54) LABISIA PUMILA EXTRACTS FOR REDUCING THE RISK OF CARDIOVASCULAR DISEASES

(75) Inventor: Wan Nazaimoon Wan Mohamud, Kuala Lumpur (MY)

Correspondence Address: WOLF GREENFIELD & SACKS, P.C., 600 ATLANTIC AVENUE, BOSTON, MA, 02210-2206, US

(73) Assignee: Government of Malaysia as represented by the Minstry of Science, Tech. and Innovation, Malaysia, Putrajaya (MY)

(21) Appl. No.: 12/867,936

(22) PCT Filed: Nov. 11, 2008
(86) PCT No.: PCT/MY08/00129

§ 371 Date: Aug. 17, 2010

(30) Foreign Application Priority Data
Mar. 19, 2008 (MY) PI 20080737

(51) Int. Cl. A61K 36/77; A61P 9/10; A61P 9/00; A61P 3/04; A61P 15/00

(52) U.S. Cl. 424/725

(57) ABSTRACT

There is disclosed a standardised aqueous extract of Labisia pumila, as a herbal medicine or as a botanical drug, that can reduce the risk or progression of cardiovascular diseases. This extract addresses factors that influence pathophysiological changes associated with insulin resistance, obesity, metabolic syndrome, diabetes and aging.

### TECHNICAL FIELD OF THE INVENTION

The present invention relates generally to the use of a standardised aqueous extract of Labisia pumila leaves prepared by the method as disclosed in Malaysian patent application number PI 20054784, as a herbal medicine or as a botanical drug, and more particularly, to reduce the risk or progression of cardiovascular diseases by addressing factors that influence the pathophysiological changes (including regulation of various related genes) associated with insulin resistance, obesity, metabolic syndrome, diabetes and aging especially in women.

### BACKGROUND OF THE INVENTION

With rapid urbanization and changing lifestyle, cardiovascular disease (CVD) is now the leading cause of death in many countries. Many factors are known to influence the degree to which an individual can develop CVD, these include age, gender, genetics and lifestyle, notably dietary habits.

Whilst healthy lifestyle habits such as exercise and a well-balanced diet are the best and most effective methods to prevent CVD, for women, estrogen has been known to play a significant role in protecting and maintaining not only the cardiovascular health but also the bone mass and mental cognition. These protective effects however, will be significantly reduced as the woman enters menopause or premature menopause a result of surgical removal of ovaries or following medical treatments such as chemotherapy and pelvic radiation therapy. On average, CVD kills five times more women than breast cancer.

Hence interventions, be it lifestyle, pharmacological or the use of complementary medicines such as herbal remedies could delay or even prevent CVD. There have been many studies that support the beneficial effects and use of dietary supplements like soy proteins, green tea and Chinese herbs like Gynostemma, Rhodiola and Ganoderma for maintaining cardiovascular health.

Labisia pumila or better known as “Kacip Fatimah” (also known as Selusoh Fatimah, Rumput Siti Fatimah, Akar Fatimah, Kachit Fatimah, Kachip Fatimah, Kachip Patimah, Kunchi Fatimah, Pokok pinggang, Rumput palls, Tadah mata hari, Mata pelandok rimba, Bunga belangkas hutan) is a Malay herb as disclosed in Burkhill (1993). Whilst cultivation is now possible, this herbaceous shrub can be found growing wildly on the forest floor only in Peninsular Malaysia and Kalimantan. For over 400 years, a decoction of the plant is traditionally taken to ease childbirth and during post-partum, to help to firm and tone vaginal and stomach muscles and to regain vitality. Additional uses include regulation of menstruation, relieve of menstrual pain, anti-flatulence, anti-dysentery and cures “sickness in the bones”.

### SUMMARY OF THE INVENTION

Accordingly, it is the primary object of the present invention to provide a use for standardised aqueous extract of Labisia pumila that can reduce the risk or progression of cardiovascular diseases and age-related health problems.

It is yet another object of the present invention to provide a use of the said standardized aqueous extract for influencing pathophysiological changes.

These and other objects of the present invention are accomplished by providing,

Use of standardised aqueous extract of Labisia pumila in orally sufficient amount characterised in that said extract reduces the risk or progression of cardiovascular diseases and age-related health problems.

and

Use of standardised aqueous extract of Labisia pumila wherein the extract influences pathophysiological changes, including regulation of genes associated with insulin resistance, obesity, metabolic syndrome, diabetes and aging.

### BRIEF DESCRIPTION OF THE FIGURES

FIG. 1 shows body weight changes in rats fed with standardised aqueous extract of Labisia pumila.

FIG. 2 shows uterine integrity of rats fed with standardised aqueous extract of Labisia pumila.

FIG. 3 shows glucose utilization of rats fed with standardised aqueous extract of Labisia pumila.

FIG. 4 shows plasma leptin level in rats fed with standardised aqueous extract of Labisia pumila.

FIG. 5 shows leptin expression in adipose tissue in rats fed with standardised aqueous extract of Labisia pumila.

FIG. 6 shows leptin expression in liver tissue in rats fed with standardised aqueous extract of Labisia pumila.

FIG. 7 shows adiponectin level in rats fed with standardised aqueous extract of Labisia pumila.

FIG. 8 shows adiponectin level in adipose tissue in rats fed with standardised aqueous extract of Labisia pumila.

FIG. 9 shows PPAR-gamma expression in adipose tissue in rats fed with standardised aqueous extract of Labisia pumila.

FIG. 10 shows PPAR-gamma expression in liver tissue in rats fed with standardised aqueous extract of Labisia pumila.

### DETAILED DESCRIPTION OF THE INVENTION

The present invention relates to the use of a standardised aqueous extract of Labisia pumila leaves prepared by the method as disclosed in Malaysian patent application number PI 20054784, as a herbal medicine or as a botanical drug, to reduce the risk or progression of cardiovascular diseases by addressing factors that influence the pathophysiological changes, including regulation of various related genes, associated with insulin resistance, obesity, metabolic syndrome, diabetes and aging especially in women.

The standardised aqueous extract of Labisia pumila leaves is not intended to replace pharmacological therapy but rather to emphasize that together with healthy lifestyle habits, the cumulative beneficial and preventive effects of this extract, especially when diligently taken, will be beneficial in maintaining health and therefore, lessen the risk of CVD and age-related health problems especially in women.

### Example 1

In one example, the effects of the extract on adipokines, glucose utilization, weight and uterine integrity in rats was studied.

Ovariectomized Sprague-Dawley rats were treated daily with standardised aqueous extract of Labisia pumila in the dose of 10 to 50 mg/kg/day for 30 days. The extract was dissolved in 2-3 ml distilled water and fed to the rats by oral gavage. At the end of the study, glucose level was determined using blood collected from the tail vein. The animals were later sacrificed and adipose fat, skeletal muscle and liver tissues excised and immediately processed for gene expression study. The plasma was stored frozen at −80° C. until analysed. Uterine weight was recorded prior to being processed for histological examination. Enzyme- or radioimmunoassay kits were used to determine the levels of insulin and adipokines hormones in the plasma samples.

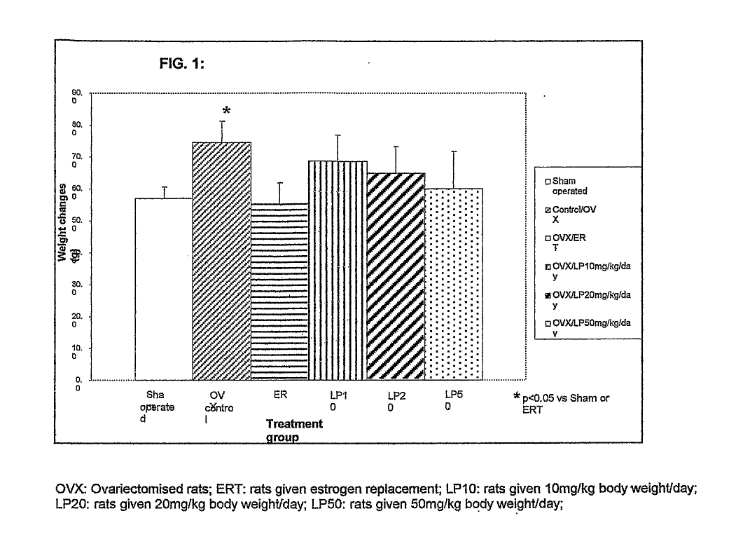

Estrogen-depleted rats showed significant increase in weight but in those given the extract, a dose-relationship was observed between the amounts of extract used and reduced weight gain (FIG. 1).

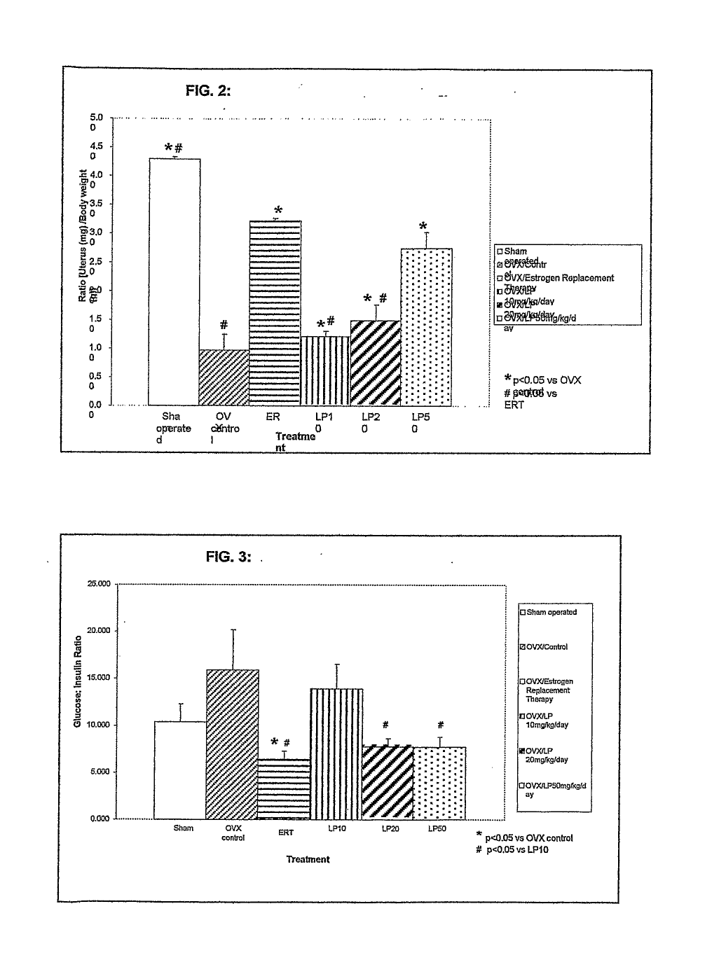

Uterine integrity of the treated animals was found to be restored with increasing dose of the extract (FIG. 2).

Compared to the control groups, animals given extract showed improved glucose utilization comparable to those on estrogen replacement (FIG. 3).

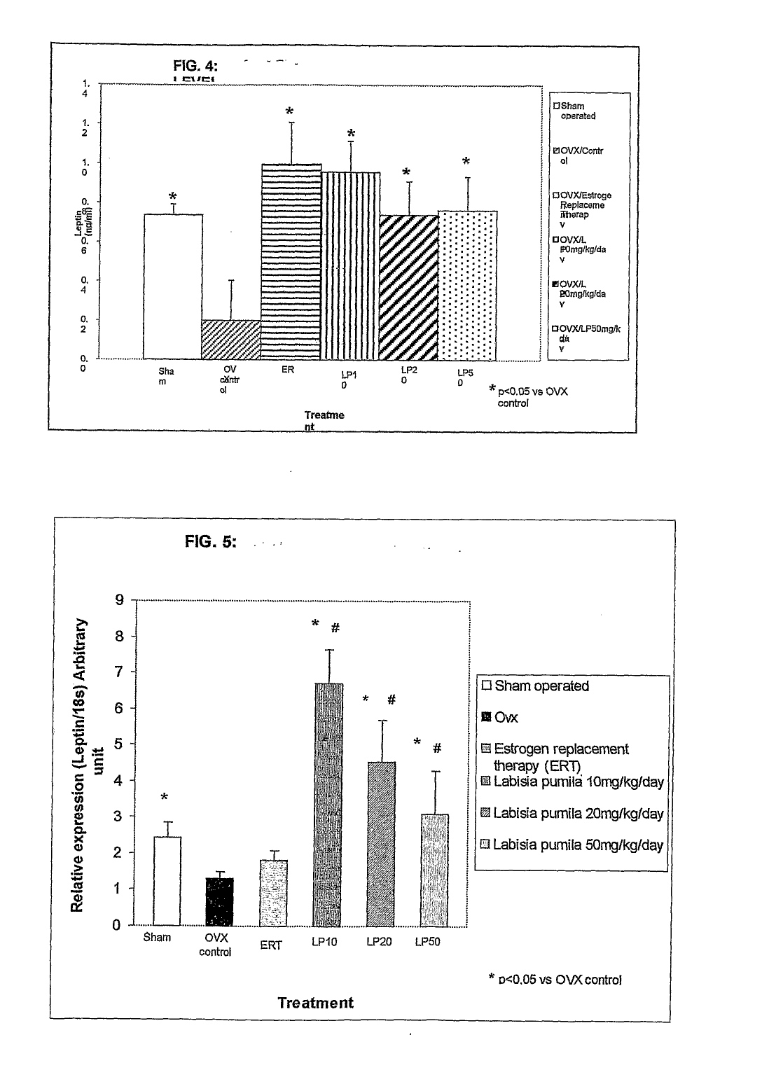

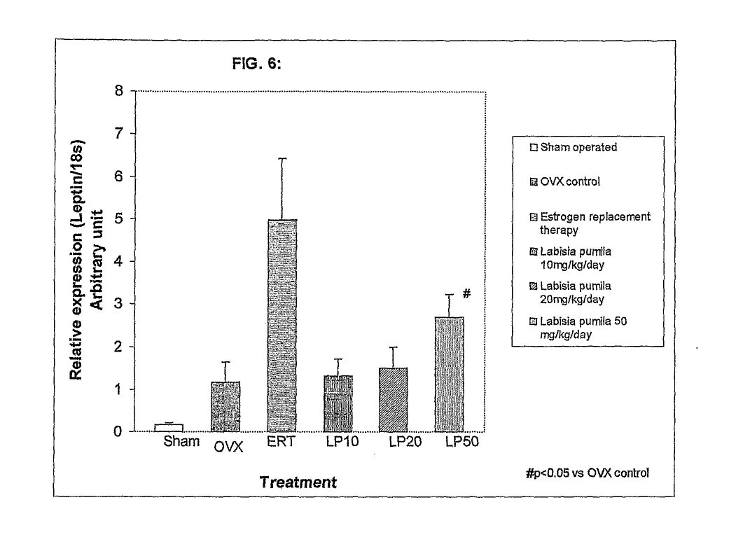

In rats given the extract, there was significant increase in plasma leptin level (FIG. 4) and leptin gene expression of the adipose (FIG. 5) and liver tissues (FIG. 6).

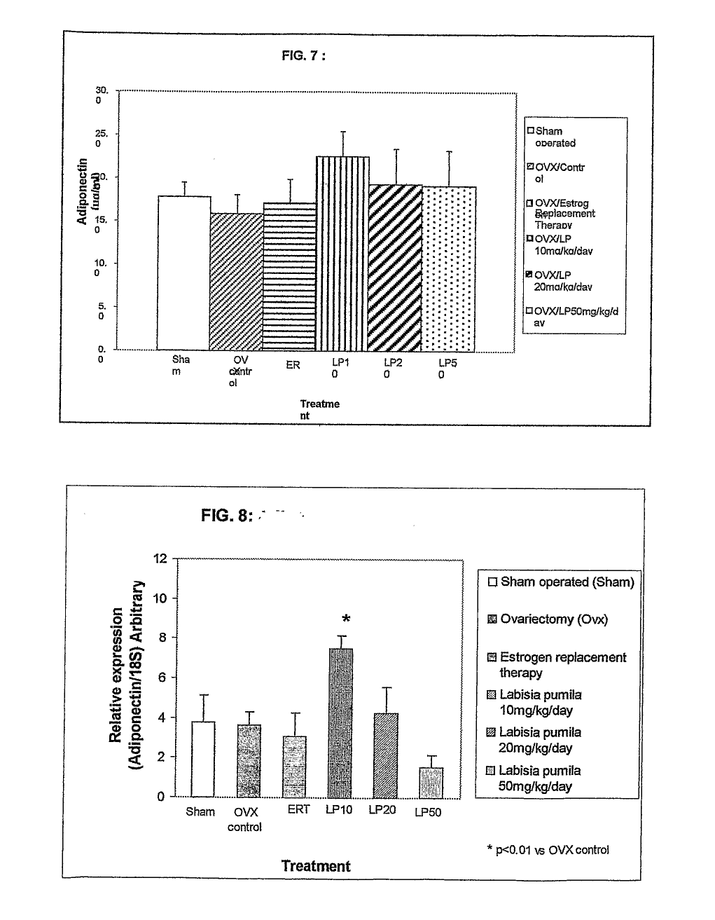

Similarly, rats fed with the extract had higher plasma adiponectin (FIG. 7) and increased adiponectin gene expression in the adipose tissues (FIG. 8).

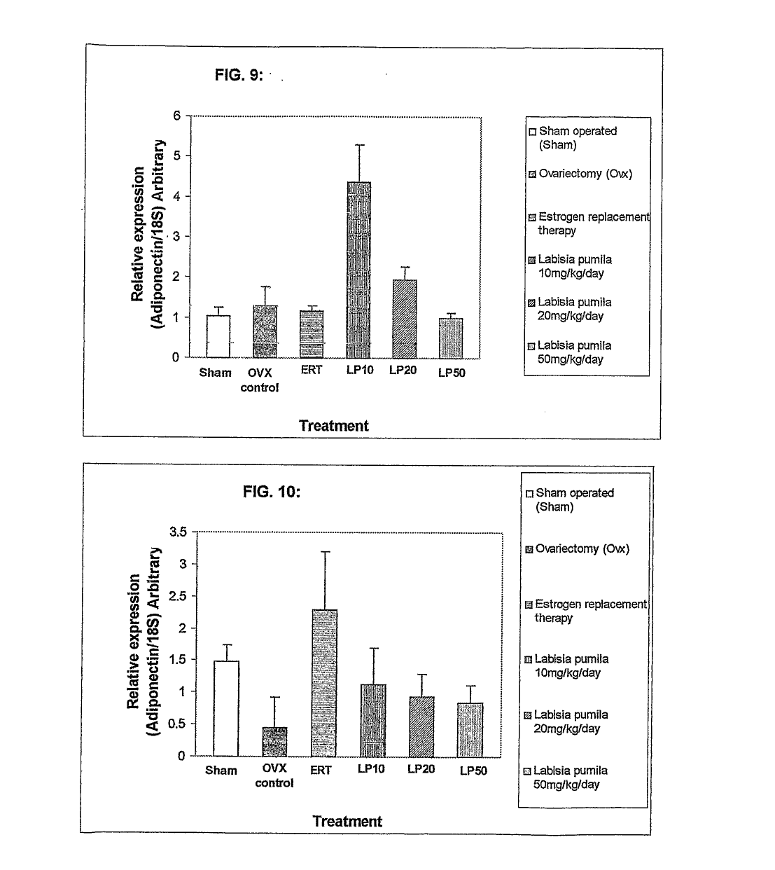

The PPAR-gamma gene expression was also higher in the adipose (FIG. 9) and liver tissues of rats given the extract (FIG. 10).

### Example 2

In another example, a double-blind placebo controlled study was carried out using 116 healthy female volunteers aged 48-55 years old. The subjects received either 140 mg or 280 or 560 mg per day of the extract or placebo for 360 days. There was an overall improvement in lipids profile, statistically significant for triglycerides (p=0.003) and Apolipoprotein B (p3=0.006) (Table 1).

TABLE 1

| | | 140 | 280 | 560 | |
|---|---|---|---|---|---|
| | Placebo | mg/day | mg/day | mg/day | P |
| Test | (n = 32) | (n = 29) | (n = 32) | (n = 25) | value |
| Total cholesterol | 5.4 | 5.5 | 5.0 | 5.0 | 0.077 |
| (mmol/L) | | | | | |
| Triglycerides | 2.02 | 1.24 | 1.30 | 1.16 | 0.003 |
| (mmol/L) | | | | | |
| HDL-cholesterol | 1.14 | 1.11 | 1.09 | 1.01 | 0.517 |
| (mmol/L) | | | | | |
| LDL-cholesterol | 3.55 | 3.73 | 3.29 | 3.41 | 0.347 |
| (mmol/L) | | | | | |
| Apolipoprotein B | 95.6 | 93.6 | 87.9 | 83.0 | 0.006 |
| (mg/dL) | | | | | |

This result further support the claim that the aqueous extract of Labisia pumila, if taken diligently, can maintain the cardiovascular health and therefore, lessen the risk of CVD and age-related health problems especially in women.

### REFERENCES

Burkill, I. H. 1993. A dictionary of the economic products of the Malay Peninsula. v. II (I-Z). Third print. Kuala Lumpur: Ministry of Agriculture. 1311.
Lerner & Kannel. Patterns in Coronary Heart Disease-Morbidity and Mortality in the Sexes: A 26-Year Follow-Up of the Framingham Population. Amer. Heart J 1986; 111: 383-390.
Richeson, L. S. et al. Relative Contributions of Aging and Estrogen Deficiency to Postmenopausal Bone Loss. N. Eng. J. Med. 1984; 311: 1273-1275.
Sayegh, R. et al. Impact of Hormone Replacement Therapy on the Body Mass and Fat Compositions of Menopausal Women: A Cross-Sectional Study, Menopause 1999; 6 (4): 312-315.
Kurzer M. S. Phytoestrogen supplement use by women. J. Nutr. 2003; 133(6): 1983-1986.
Michael Blaha and Tom A Elasy. Clinical use of the metabolic syndrome: why the confusion? Clin Diab 2006; 24: 125-131
Reaven G. The metabolic syndrome or the insulin resistance syndrome? Different names, different concepts, and different goals. Endocrinol Metab Clinics North Am 2004; 33:283-303.
Abel Romero-Corral et al. Association of bodyweight with total mortality and with cardiovascular events in coronary artery diseases: a systematic review of cohort studies. Lancet 2006; 368: 666-678

It will of course be realised that whilst the above has been by way of an illustrative example of the invention, all such and other modifications and variations thereto, as would be apparent to persons skilled in the art, are deemed to fall within the broad scope and ambit of the invention as claimed herein.

1. A standardised aqueous extract of Labisia pumila in orally sufficient amount for use in reducing the risk or progression of cardiovascular diseases and age-related health problems.

2. The standardised aqueous extract as claimed in claim 1, wherein the amount of extract orally taken ranges from 100-500 mg/day.

3. The standardised aqueous extract as claimed in claim 1, wherein the extract influences pathophysiological changes, including regulation of genes associated with insulin resistance, obesity, metabolic syndrome, diabetes and aging.

4. The standardised aqueous extract as claimed in claim 2, wherein the 100-500 mg/day of the extract causes lowering of biochemical markers for lipids, apolipoproteins and endothelial functions.

5. The standardised aqueous extract as claimed in claim 1 in an amount of 10-50 mg/kg body weight/day, for use in regulating the level of adipokines and related gene expression in mammals.

6. The standardised aqueous extract as claimed in claim 1 in an amount of 10-50 mg/kg body weight/day, for use in improving glucose utilization and regulating body weight in mammals.

7. The standardised aqueous extract as claimed in claim 1 in an amount of 10-50 mg/kg body weight/day, for use in maintaining uterine wall integrity in mammals.

8. The standardised aqueous extract as claimed in claim 1, for use in reducing the risk of cardiovascular diseases and age-related health problems, especially in women.

9. A method for reducing the risk or progression of cardiovascular diseases and age-related health problems comprising administering to a subject an orally sufficient amount of a standardised aqueous extract of Labisia pumila to reduce the risk or progression of cardiovascular diseases and age-related health problems.

10. The method of claim 9, wherein the amount of extract orally administered ranges from 100-500 mg/day.

11. The method of claim 9, wherein the extract influences pathophysiological changes, including regulation of genes associated with insulin resistance, obesity, metabolic syndrome, diabetes and aging.

12. The method of claim 10, wherein the 100-500 mg/day of the extract causes lowering of biochemical markers for lipids, apolipoproteins and endothelial functions.

13. The method of claim 9, wherein the extract is administered in an amount of 10-50 mg/kg body weight/day, wherein the extract regulates the level of adipokines and related gene expression in mammals.

14. The method of claim 9, wherein the extract is administered in an amount of 10-50 mg/kg body weight/day, wherein the extract improves glucose utilization and regulates body weight in mammals.

15. The method of claim 9, wherein the extract is administered in an amount of 10-50 mg/kg body weight/day, wherein the extract maintains uterine wall integrity in mammals.

16. The method of claim 9, wherein the extract reduces the risk of cardiovascular diseases and age-related health problems, especially in women.
